SEQUENCE LISTING (4 sequences)

SEQ ID NO: 1 - DNA, 29 bp, Homo sapiens
gtcacattttcattatttttattataagg

SEQ ID NO: 2 - DNA, 22 bp, Homo sapiens
gtatcgtcaaggcactcttgcc

SEQ ID NO: 3 - DNA, 16 bp, Homo sapiens
ttggagctggtggcgt

SEQ ID NO: 4 - DNA, 15 bp, Homo sapiens
ctggtgacgtaggca

(19) United States
(12) Patent Application Publication
(10) Pub. No.: US 2022/0364164 A1
(43) Pub. Date: Nov. 17, 2022

(54) DIGITAL PCR MEASUREMENT METHOD AND MEASUREMENT DEVICE

(71) Applicant: Hitachi, Ltd., Tokyo (JP)

(72) Inventors: Junko TANAKA, Tokyo (JP); Tatsuo NAKAGAWA, Tokyo (JP); Yuzuru SHIMAZAKI, Tokyo (JP); Akiko SHIRATORI, Tokyo (JP)

(21) Appl. No.: 17/620,077

(22) PCT Filed: May 27, 2020
(86) PCT No.: PCT/JP2020/020998

§ 371 Date: Dec. 16, 2021

(30) Foreign Application Priority Data
Jun. 26, 2019 (JP) 2019-118981

(51) Int. Cl. C12Q 1/6851; B01L 7/00; B01L 3/00

(52) U.S. Cl. CPC C12Q 1/6851; B01L 7/52; B01L 3/5027; C12Q 1/6818

(57) ABSTRACT

The purpose of the present invention is to provide a novel digital PCR analysis method. In the digital PCR analysis method disclosed herein, a method for detecting DNA is used, which includes the steps of: dividing a DNA solution containing a fluorescent-labeled probe or a DNA intercalator and a plurality of DNAs to be detected into a plurality of compartments; carrying out PCR in the compartments; measuring a fluorescence intensity in association with a change in temperature; calculating a melting temperature from a melting curve for a DNA double strand measured on the basis of a change in fluorescence intensity, which is associated with the change in temperature; and calculating a temperature difference between two points with a slope of a predetermined value on a melting curve indicating a change in the fluorescence intensity.

### TECHNICAL FIELD

The present invention relates to a digital PCR measurement method and measurement device.

### BACKGROUND ART

Digital PCR (JP 2013-521764 W) has been developed as a method for solving the problem that measurement reproducibility is deteriorated when the amount of a gene to be detected (herein, referred to as a target gene) is minute in a conventional genetic test such as PCR (U.S. Pat. Nos. 4,683,195; 4,683,202; or U.S. Pat. No. 4,800,159) or real-time PCR (Genome Res., 10, pp 986-994, 1996). When digital PCR is used, a minute amount of DNA can be quantified by detecting DNA on a 0 (none) or 1 (presence) basis using a sample subjected to limiting dilution.

An example of a digital PCR detection method is shown below. First, a PCR reaction solution is prepared by adding a DNA polymerase, a primer and a fluorescent-labeled probe necessary for PCR to a specimen subjected to limiting dilution. The PCR reaction solution is divided into minute compartments such as wells or droplets. Here, one molecule of the target gene is present or is not present in one compartment. Next, the target gene in the minute compartment is amplified by PCR. The target gene can be quantified by measuring the fluorescence intensity of each minute compartment after PCR, and counting the number of minute compartments having a fluorescence intensity exceeding the threshold.

In such digital PCR, a specimen subjected to limiting dilution is used, and therefore it is possible to suppress impacts of a component derived from the specimen which is a factor of inhibiting a reaction in PCR. In addition, since a calibration curve is not required, the absolute amount of target DNA can be directly measured.

Meanwhile, in conventional PCR, it is known that reaction efficiency decreases because of the presence of a reaction inhibitor in a reaction solution, formation of a secondary structure of template DNA, insufficient design of a primer, and the like.

On the other hand, in digital PCR, measurement is performed at the end point of the reaction, and therefore it has been considered that the reaction efficiency itself of PCR does not significantly affect measurement results. In reality, however, even when measurement is performed at the end point, the fluorescence intensity significantly varies because the minute compartments are not uniform in PCR reaction efficiency, and thus measurement reproducibility and measurement accuracy of digital PCR are deteriorated.

Thus, for improving measurement reproducibility and measurement accuracy of digital PCR, the present inventors have developed a technique capable of discriminating a target gene in a minute compartment by measuring a melting temperature (Tm) of a PCR amplicon even if minute compartments are not uniform in PCR reaction efficiency (JP 2018-108063 A). Specifically, by, for example, measuring the melting temperatures (Tm) of a target gene amplified in a minute compartment and a fluorescent-labeled probe after PCR, the genotype of the target gene can be identified by a difference in melting temperature even if the reaction efficiency of PCR is not uniform.

### SUMMARY OF INVENTION

### Technical Problem

In digital PCR, the number of target genes present in a minute compartment follows the Poisson distribution, and therefore when the specimen is diluted, two molecules of the target gene are present in one compartment with a certain probability although mostly, one molecule of the target gene is present or is not present in one compartment. Two molecules of a mutant which is less common are rarely present in the same compartment, but presence of one molecule of a mutant and one molecule of a wild-type in the same compartment can easily occur. It is important to discriminate such a minute compartment containing two types of molecules for reducing false-negative cases and false-positive cases of mutant genes and improving measurement reproducibility and measurement accuracy.

Thus, an object of the present invention is to provide a novel digital PCR measurement method and measurement device for clearly discriminating minute compartments, in which two different types of genes to be detected are present in one compartment, by a measurement device and correcting the count number of target genes in digital PCR using melting curve analysis.

### Solution to Problem

The present inventors have found that in digital PCR using melting curve analysis, there are two types of target genes different in melting temperatures from probes, and when the probes are labeled with the same fluorescent dye, the slope of the melting curve becomes gentle as a whole and the FWHM (full width at half maximum) of the differential curve of the melting curve increases when one molecule of each of the two types of target genes is present in the same minute compartment, so that minute compartments containing two types of molecules can be discriminated by calculating the FWHM (full width at half maximum) in addition to the melting temperature from the differential curve of the melting curve, leading to completion of the present invention.

One embodiment of the present invention is a method for detecting DNA, including the steps of: dividing a DNA solution containing a fluorescent-labeled probe or a DNA intercalator and a plurality of types of DNAs to be detected into a plurality of minute compartments; carrying out PCR in the minute compartments; measuring a fluorescence intensity in association with a change in temperature; calculating a melting temperature of a DNA double strand from a change in fluorescence intensity, which is associated with a change in temperature of the DNA solution; and calculating a temperature difference between two points with a slope of a predetermined value on a melting curve indicating a change in the fluorescence intensity. The method may further include the step of identifying a compartment, in which the temperature difference is equal to or greater than a predetermined threshold, as a compartment containing two types of the DNAs to be detected, and the step of identifying a compartment, in which the temperature difference is less than the predetermined threshold, as a compartment containing one type of the DNA to be detected.

In any one of the methods for detecting DNA, the DNA solution may contain a fluorescent-labeled probe, and the melting temperature may be a melting temperature of a double strand formed between the fluorescent-labeled probe and the DNA to be detected. Here, the fluorescent-labeled probe may have a fluorescent dye and a quencher thereof. Alternatively, the DNA solution may contain a DNA intercalator, and the melting temperature may be a melting temperature of the double strand DNA to be detected.

In any of the methods for detecting DNA, the plurality of minute compartments may be arranged in a plane. The DNA solution may be divided into the plurality of compartments by droplets or wells.

Another embodiment of the present invention is a DNA detector for detecting DNAs in a DNA solution containing a plurality of types of DNAs to be detected, the DNA detector including: a heating unit for heating the DNA solution; a fluorescence measuring unit for measuring an intensity of fluorescence emitted from the DNA solution; and a calculation unit for calculating a melting temperature of a DNA double strand from a change in intensity of the fluorescence, which is associated with a change in temperature of the DNA solution, and calculating a temperature difference between two points with a slope of a predetermined value on a melting curve indicating the change in the fluorescence intensity. The DNA detector may further include an amplification unit for amplifying the DNA to be detected. In addition, the DNA detector may further include a monitor which displays a detection result.

A further embodiment of the present invention is a program for causing a DNA detector such as any of the DNA detectors to carry out any of the methods for detecting DNA.

A further embodiment of the present invention is a recording medium which stores the program.

==Cross-Reference to Related Documents==

The present application claims priority based on Japanese Patent Application No. 2019-118981 filed on Jun. 26, 2019, which is incorporated herein by reference to the basic application.

### BRIEF DESCRIPTION OF DRAWINGS

FIG. 1 is a diagram showing a basic concept of a method for detecting DNA by calculating a temperature difference between two points with a slope of a predetermined value on a melting curve showing a change in fluorescence intensity in addition to a melting temperature of a DNA double strand measured on the basis of a change in fluorescence intensity, which is associated with a change in temperature, on a PCR amplicon, in digital PCR using melting curve analysis in one embodiment of the present invention.

FIG. 2 is a diagram showing a basic concept of a method for detecting DNA using a melting temperature and a fluorescence intensity of a PCR amplicon in digital PCR using melting curve analysis in one embodiment of the present invention.

FIG. 3 is a schematic diagram of a fluorescence measuring unit for measuring the color and fluorescence intensity of a fluorescent dye contained in a droplet or a well in one embodiment of the present invention.

FIG. 4 shows (A) an example of a result of digital PCR measurement using a method for detecting DNA using a fluorescence intensity and a melting temperature of a PCR amplicon and (B) an example of a result of digital PCR measurement using a method for detecting DNA using a temperature difference between two points with a slope of a melting curve of a predetermined value and a melting temperature in a differential function of a change in fluorescence intensity on the PCR amplicon, in an embodiment of the present invention.

FIG. 5 is a schematic diagram showing a method for measuring a melting temperature of DNA using a DNA intercalator in a method for detecting DNA in one embodiment of the present invention.

FIG. 6 is a schematic diagram showing a method for measuring a melting temperature of DNA using a fluorescent-labeled probe in a method for detecting DNA in one embodiment of the present invention.

FIG. 7 is a flowchart showing one embodiment of a method for measuring a melting temperature using the device and the cartridge of FIG. 3.

FIG. 8 is an example of a measurement result displayed on a monitor.

FIG. 9 is an example of a measurement result displayed on a monitor.

FIG. 10 is a graph showing a result of discriminating the type of a target gene in a well using a fluorescent-labeled probe in an example of the present invention.

### DESCRIPTION OF EMBODIMENTS

Objects, features, advantages and ideas of the present invention will be apparent to those skilled in the art from the description herein, and those skilled in the art can easily reproduce the present invention from the description herein. The embodiments and specifically examples of the invention described below indicate preferred embodiments of the present invention, are shown for the purpose of illustration or description, and do not limit the present invention. It will be apparent to those skilled in the art that various changes and modifications can be made on the basis of the description herein within the spirit and scope of the invention disclosed herein.

(1) Principle and Effect of Method for Detecting DNA A method for detecting DNA according to the present invention includes the steps of: dividing a DNA solution containing a fluorescent-labeled probe or a DNA intercalator and a plurality of types of DNAs to be detected into a plurality of compartments; carrying out PCR in the compartments; measuring a fluorescence intensity in association with a change in temperature; calculating a melting temperature of a DNA double strand from a change in fluorescence intensity, which is associated with a change in temperature of the DNA solution; and calculating a temperature difference between two points with a melting curve slope of a predetermined value on a melting curve indicating a change in the fluorescence intensity. The slope of the melting curve at a certain point on the melting curve means a slope of a tangent to the melting curve at that point.

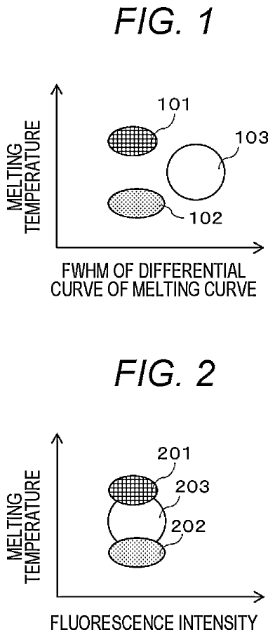

Here, FIG. 1 shows an example of a measurement result assumed in a typical embodiment of a method for detecting DNA by calculating a temperature difference between two points with a slope of a predetermined value on a melting curve showing a change in fluorescence intensity in addition to a melting temperature of a DNA double strand measured on the basis of a change in fluorescence intensity, which is associated with a change in temperature, on a PCR amplicon. FIG. 2 shows a measurement result of digital PCR using melting curve analysis which is carried out using a melting temperature and a fluorescence intensity of a PCR amplicon.

In digital PCR using melting curve analysis, genotypes are discriminated by making use of the fact that the melting temperatures of a fluorescent-labeled probe and DNA vary depending on the genotype. The example of FIG. 2 schematically shows the results of measuring the melting temperature of DNA in each minute compartment using a fluorescent-labeled probe corresponding to each of the wild-type and mutant of the target gene. Here, as the fluorescent-labeled probe, for example, a molecular beacon can be used, and hereinafter, the method for detecting DNA will be described in detail with a molecular beacon taken as an example. The molecular beacon is an oligonucleotide which is complementary to a sequence between a pair of primers used in PCR for amplifying a gene to be detected, has complementary sequences at both ends, and has a fluorescent dye and a quenching dye (quencher) provided at an end. When the molecular beacon is hybridized with the gene to be detected, the fluorescent dye and the quenching dye at both ends are separated from each other to emit fluorescence, and when the molecular beacon is separated from the gene to be detected as the temperature rises, complementary sequences at both ends are hybridized to form a stem-loop structure, and the fluorescent dye and the quenching dye approach each other to quench the fluorescent dye. In a minute compartment 201 containing a wild-type allele of the gene to be detected, a fluorescent-labeled probe corresponding to the wild-type allele of the gene to be detected is hybridized with DNA amplified by PCR to emit fluorescence, so that a melting temperature corresponding to the fluorescent-labeled probe of the wild-type allele is observed. In a minute compartment 202 containing a mutant allele of the gene to be detected, a fluorescent-labeled probe corresponding to the mutant allele of the gene to be detected is hybridized with DNA amplified by PCR to emit fluorescence, so that a melting temperature corresponding to the fluorescent-labeled probe of the mutant allele is observed. Thus, whether a gene to be detected, which has a wild-type allele is present or not and whether a gene to be detected, which has a mutant allele is present or not can be determined by the fluorescence intensity, the type of fluorescence and the melting temperature. It may be difficult to discriminate between the minute compartment 201 containing a wild-type allele and the minute compartment 202 containing a mutant allele of the gene to be detected, by the fluorescence intensity because the minute compartments are not uniform in reaction efficiency of PCR in the minute compartment and there is a significant planar measurement variation during fluorescence measurement. Even in such a case, the melting temperature of DNA is not influenced by the reaction efficiency of PCR or the planar measurement variation during fluorescence measurement, by determining the sequence of fluorescent-labeled probes in such a manner that the melting temperature (Tm) of each fluorescent-labeled probe is different from that of the gene to be detected, measuring a fluorescence intensity change associated with a change in temperature on DNA in the minute compartment, performing melting curve analysis, and comparing the melting temperatures, a gene can be more accurately detected.

Thus, in digital PCR, an experimenter can set threshold values of the fluorescence intensity and the melting temperature, exclude empty minute compartments free of a target gene from the data, and count the number of minute compartments for each type of mutation. However, in digital PCR, the number of target genes present in a minute compartment follows the Poisson distribution, and therefore when the specimen is diluted, two molecules of the target gene are present in one compartment with a certain probability although mostly, one molecule of the target gene is present or is not present in one compartment. In a minute compartment 203 containing one molecule of each of a wild-type allele and a mutant allele of the gene to be detected, fluorescent-labeled probes corresponding, respectively, to the wild-type allele and the mutant allele of the gene to be detected are hybridized with DNA amplified by PCR to emit fluorescence, so that a temperature intermediate between melting temperatures corresponding to the wild-type allele and the mutant allele is observed. However, when the melting temperature of each fluorescent-labeled probe is not sufficiently different from that of the gene to be detected, e.g. the difference therebetween is 10° C. or less, preferably 5° C. or less, more preferably 3° C. or less, still more preferably 1° C. or less, and more than 0° C., two melting curves from the wild-type allele and the mutant allele of the gene to be detected are combined and observed as one melting curve with a small slope, and therefore the differential curve for calculating the melting temperature has a large shape, so that the melting temperature is difficult to fix, leading to an increase in variation. As a result, as shown in FIG. 2, the distributions on the graph of the minute compartment 201 containing the wild-type allele of the gene to be detected and the minute compartment 202 containing the mutant allele of the gene to be detected overlap the distribution on the graph of the minute compartment 203 containing one molecule of each of the wild-type allele and the mutant allele of the gene to be detected, and at the overlapped portion, whether the gene is present or not cannot be determined. This causes a decrease in measurement accuracy.

Thus, by making use of the fact that when one molecule of each of two types of genes to be detected is present in the same minute compartment, the slope of the melting curve decreases and the temperature difference between the two points with a slope of a predetermined value on the melting curve increases, the temperature difference between the two points with a slope of a predetermined value on the melting curve in a differential curve of the melting curve is calculated in addition to the melting temperature. When the measurement results are plotted where the abscissa represents a temperature difference between two points with a slope of a predetermined value on the melting curve and the ordinate represents the melting temperatures of the fluorescent-labeled probe and DNA, the distributions of the minute compartment 201 containing the wild-type allele of the gene to be detected, the minute compartment 202 containing the mutant allele of the gene to be detected and the minute compartment 203 containing one molecule of each of the wild-type allele and the mutant allele of the gene to be detected can be separated on the graph. Here, the value used for the abscissa may be one indicating the shape of the melting curve or the differential curve of the melting curve, and is preferably a temperature difference between two points with a slope of a predetermined value on the melting curve, more preferably a FWHM (full width at half maximum) of the differential curve. By using the shape of the melting curve for discrimination of a gene to be detected, which is amplified in a minute compartment, discrimination can be reliably performed even if two types of genes to be detected are present in the same minute compartment, and it is possible to improve measurement reproducibility and measurement accuracy.

(2) Principal Configuration of DNA Detector

The DNA detector of the present invention is a DNA detector for detecting DNA to be detected in a DNA solution, and includes: a heating unit for heating the DNA solution; a fluorescence measuring unit for measuring an intensity of fluorescence emitted from the DNA solution; and a calculation unit for calculating a melting temperature of a DNA double strand from a melting curve representing a change in intensity of the fluorescence, which is associated with a change in temperature of the DNA solution, and calculating a shape of the melting curve or the differential curve of the melting curve.

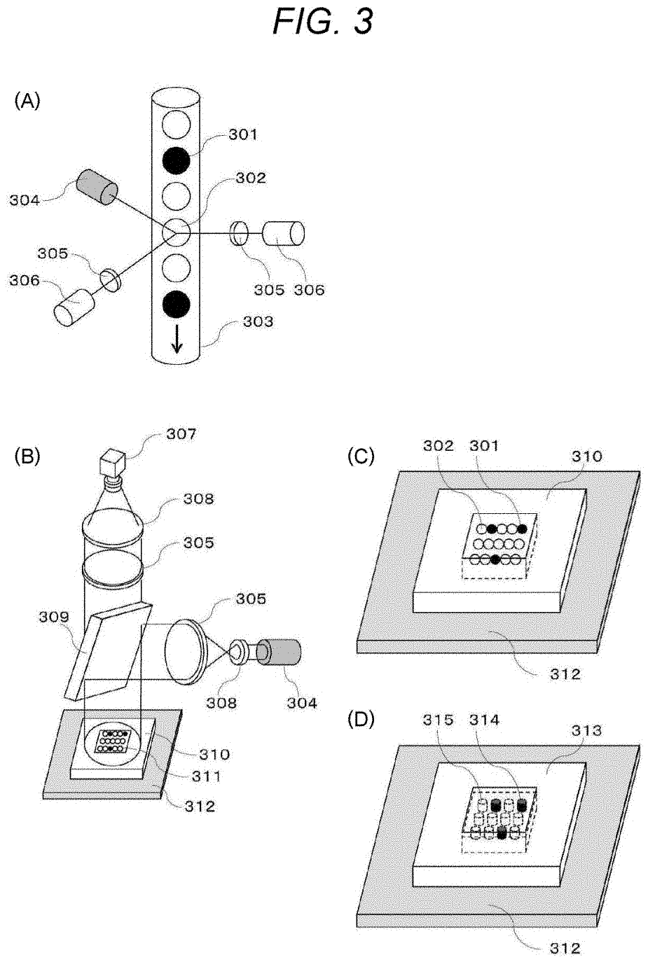

The DNA solution may be present in any carrier, and may be, for example, a droplet in oil or a solution in a well such as a plate. As an example of the DNA detector, FIG. 3 shows a DNA detector including a fluorescence measuring unit for measuring the color and the fluorescence intensity of a fluorescent dye contained in a DNA solution in a droplet or a well, but the DNA detector of the present invention is not limited thereto.

In the example of the fluorescence measuring unit shown in FIG. 3A, the fluorescence intensity of a droplet is measured using a microchannel. A droplet 301 flows in a direction of an arrow in a microchannel 303. When the droplet flows to the position of a droplet 302, the droplet is heated by a heating unit (not shown), while the droplet is irradiated with excitation light by a light source 304. The fluorescent substance contained in the droplet is excited by the light source 304, and the emitted fluorescence is detected by a photomultiplier 306 through a fluorescence filter 305. The detected fluorescence data is sent to a calculation unit (not shown), where the melting temperatures of the fluorescent-labeled probe and DNA or the melting temperature of double strand DNA is calculated. The fluorescence measuring unit including the light source 304, the fluorescence filter 305 and the photomultiplier 306 may be separately provided for each color of the fluorescent dye, or may be configured to perform excitation with excitation light from one light source and detect the fluorescences by two fluorescence filters at a time as shown in FIG. 3A.

In addition, as shown in FIGS. 3B and 3C, droplets may be arranged in a plane, followed by measuring the color and the fluorescence intensity of the fluorescent dye of each of the droplets may be measured. Specifically, for example, the droplets 311 are arranged in a plane in a droplet detection cartridge 310, and set on a temperature control stage 312 which is a heating unit. The temperature of the droplet detection cartridge is changed by the temperature controller 312, and the fluorescence intensity of the droplet associated with the change in temperature is measured in accordance with the following procedure. First, the droplets 311 arranged in a plane in the droplet detection cartridge 310 are irradiated with the excitation light from the light source 304 through a lens 308, a filter 305 and a dichroic mirror 309. The fluorescent substance contained in the droplet is excited by the excitation light, and emitted fluorescence is detected through the dichroic mirror 309, the filter 305 and the lens 308 by a CCD camera 307. The detected fluorescence data is sent to a calculation unit (not shown), where the melting temperature of the amplicon is calculated. In FIG. 3A, it is necessary to treat droplets one by one, and the device in FIGS. 3B and 3C is preferable because a large number of droplets can be processed at one time. The device in FIGS. 3B and 3C is more suitable than that in FIG. 3A because the temperature controller 312 can also be used for DNA amplification reaction.

Further, with the use of wells arranged in an array form as in FIG. 3D in stead of droplets, a specimen may be added in such a manner that one target gene or no target gene is present in one well, followed by performing PCR in the wells to measure the color and the fluorescence intensity of the fluorescent dye in the wells. Specifically, for example, after a reaction solution containing a specimen is added to wells provided in a well-type detection cartridge 313, PCR is performed in the wells, and the wells are set on the temperature control stage 312 which is a heating unit. The temperature of the well-type detection cartridge is changed by the temperature controller 312, and the fluorescence intensity of the well associated with the change in temperature is measured in accordance with the following procedure. First, the wells arranged in a plane in the well-type detection cartridge 313 are irradiated with the excitation light from the light source 304 through the lens 308, the filter 305 and the dichroic mirror 309. The fluorescent substance contained in the reaction solution in the well is excited by the excitation light, and emitted fluorescence is detected through the dichroic mirror 309, the filter 305 and the lens 308 by a CCD camera 307. The detected fluorescence data is sent to a calculation unit (not shown), where the melting temperature of the amplicon is calculated. When the well is used as in FIG. 3D, processes ranging from PCR to melting curve analysis can be carried out in the well-type detection cartridge without a step of arranging droplets in the droplet detection cartridge.

The DNA detector according to one embodiment of the present invention may include a sample dividing unit for dividing a DNA solution containing DNA to be detected into minute compartments such as wells arranged on an array in a cartridge or droplets dispersed in oil, and/or an amplification unit for amplifying DNA with respect to the minute compartments.

(3) Melting Curve Analysis Method

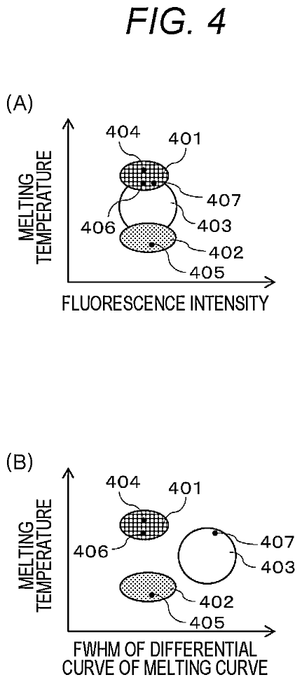

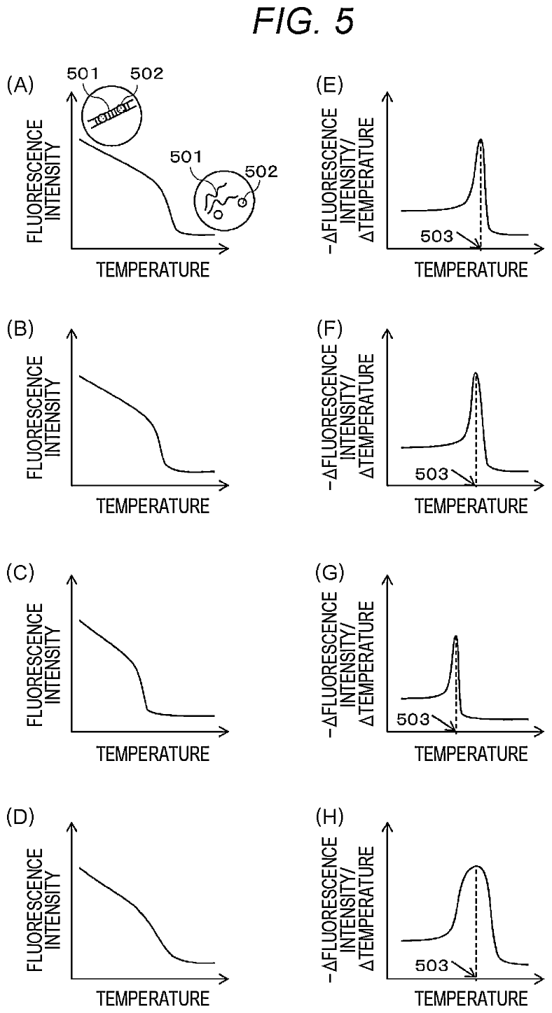

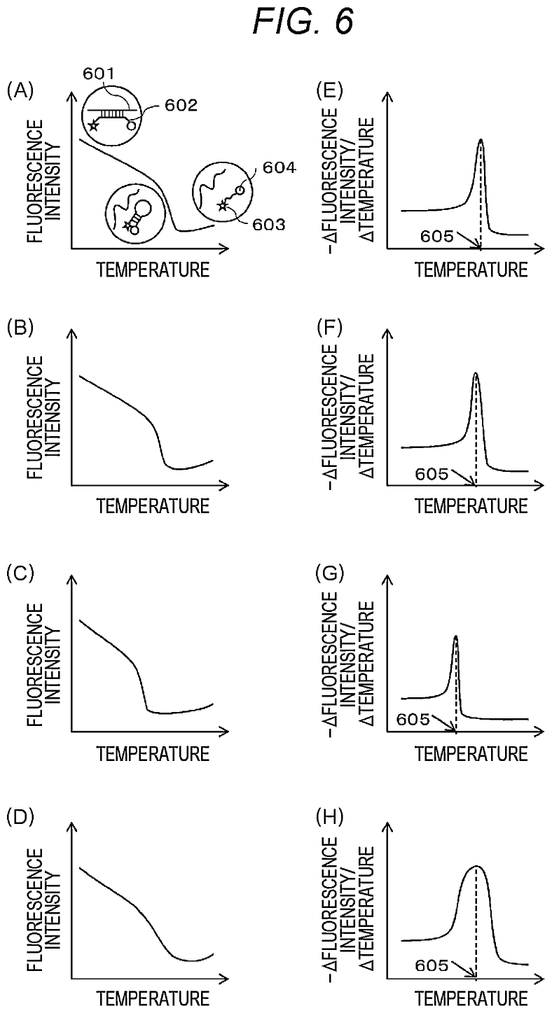

FIG. 4A is a schematic diagram showing an example of a measurement result in which as described in FIG. 2, there is a case where the measured melting temperatures overlap each other and thus it is not possible to determine the type of the target gene in the specimen solution in each minute compartment when a method for detecting DNA using the melting temperature (Tm) of a PCR amplicon. On the other hand, FIG. 4B is a schematic diagram showing an example of a result of digital PCR measurement using a method for detecting DNA using a temperature difference between two points with a slope of a predetermined value on a melting curve showing a change in fluorescence intensity, and a melting temperature, on a PCR amplicon, where it is possible to more accurately determine the type of a target gene in a specimen solution in each minute compartment. FIGS. 5 and 6 are schematic diagrams showing an example of a result of melting curve analysis of DNA amplified in a solution, on a specimen solution in which the type of a gene to be detected cannot be determined in FIG. 4.

As shown in FIG. 4A, when genotype of a gene to be detected in a specimen solution is determined by a melting temperature and a fluorescence intensity at a low temperature, it can be seen from the value of the melting temperature that a solution a404 contains a wild-type gene of the gene to be detected and a specimen solution b405 contains a mutant gene of the gene to be detected when the measurement result is plotted at the position of the specimen solution a404 or the specimen solution b405. However, when the fluorescence intensity is observed at the position of a specimen solution c406 or a specimen solution d407, whether or not such a solution contains a mutant of the gene to be detected cannot be determined from the measurement result.

Thus, a temperature difference between two points with a slope of a predetermined value on the melting curve showing a change in fluorescence intensity is also calculated in measurement of the melting temperature of DNA amplified in the specimen solution using a DNA intercalator, whereby it is possible to determine whether a target gene is present or not, which cannot be determined in FIG. 4A. As a specific method, first, a DNA intercalator 502 is added to a PCR reaction solution to prepare a specimen solution, and PCR is carried out. Consequently, approximately at room temperature, the DNA intercalator 502 is bound to double-stranded DNA 501 amplified in the specimen solution, so that intense fluorescence is emitted. Thereafter, with a rise in temperature of the specimen solution, the double-stranded DNA 501 in the specimen solution is dissociated into a single-stranded DNA 501, so that the DNA intercalator 502 is not bound, and therefore the fluorescence intensity decreases. FIG. 5 shows an example of a result when a change in fluorescence intensity change with respect to the change in temperature here is plotted on a graph. Measurement of a change in fluorescence intensity with respect to a change in temperature may be performed by raising the temperature of the specimen solution independently of PCR (e.g., after completion of PCR).

In FIG. 5, the measurement result for a specimen solution a404 is shown in FIG. 5A, the measurement result for a specimen solution b405 is shown in FIG. 5C, the measurement result for a specimen solution c406 is shown in FIG. 5B, and the measurement result for a specimen solution d407 is shown in FIG. 5D. Further, when the fluorescence intensity changes in FIGS. 5A to 5D are differentiated by change in temperatures, the results shown in FIGS. 5E to 5H are obtained, respectively, and temperatures as inflection points are determined, and can be calculated as the melting temperatures of the DNA double strands. In FIG. 4A, whether or not the specimen solution contains a target gene cannot be determined from the measurement result for the specimen solution c406 and the specimen solution d407, but since the slope of the melting curve is large in FIG. 5B and small in FIG. 5D, the temperature difference between two points with a slope of a predetermined value on the melting curve (=−Δ fluorescence intensity/Δ temperature) in the differential curve of the melting curve is small in FIG. 5F and large in FIG. 5H, and when the temperature difference between two points with a slope of a predetermined value on the melting curve is plotted on the abscissa and the melting temperature is plotted on the ordinate as in FIG. 4B, it can be determined that the specimen solution c406 is a solution containing a wild-type of a target gene and the specimen solution d407 can is a solution containing both a wild-type and a mutant.

The melting temperature of the target gene can be controlled depending on the sequence of the PCR amplicon and the strand length of the sequence by changing the design of the primer.

The DNA intercalator used here can be applied as long as it is an intercalator that is bound to double-stranded DNA to increase fluorescence intensity and can be used for detection of double-stranded DNA. Specifically, SYBR (registered trademark) Green I, SYBR Gold, PicoGreen (registered trademark), SYTO (registered trademark) Blue, SYTO Green, SYTO Orange, SYTO Red, POPO (registered trademark)-1, BOBO (registered trademark)-1, YOYO (registered trademark)-1, TOTO (registered trademark)-1, JOJO (registered trademark)-1, POPO-3, LOLO (registered trademark)-1, BOBO-3, YOYO-3, TOTO-3, PO-Pro (registered trademark)-1, YO-Pro (registered trademark)-1, TO-Pro (registered trademark)-1, JO-Pro (registered trademark)-1, PO-Pro-3, YO-Pro-3, TO-Pro-3, TO-Pro-5, ethidium bromide and the like can be applied. When the DNA intercalator has heat resistance, the DNA intercalator can be added to the well or droplet before the PCR reaction is carried out.

In this method, it is also possible to use a fluorescent-labeled probe instead of the DNA intercalator as shown in FIG. 6. The fluorescent-labeled probe is designed so as to have a fluorescent dye and a quencher thereof at or near both ends, complementary sequences on the periphery of both ends, form a stem-loop structure like a molecular beacon, and have a structure in which the sequence of a loop portion is complementary to a gene to be detected, and can be hybridized with the gene to be detected. When a fluorescent-labeled probe 602 is present alone in a free form, a stem-loop is formed, and a fluorescent dye 603 and the quencher 604 are close to each other, so that fluorescence is not emitted. When the fluorescent-labeled probe 602 is added to the specimen solution in which the PCR reaction has been completed, the loop portion of the fluorescent-labeled probe 602 is annealed to DNA 601 amplified in the specimen solution approximately at room temperature, and the fluorescent dye 603 and the quencher 604 are separated, so that the fluorescent-labeled probe 602 emits intense fluorescence. When the specimen solution is then heated, DNA 601 and the fluorescent-labeled probe 602 are separated, and a stem-loop is formed in the fluorescent-labeled probe 602, so that the fluorescence intensity from the fluorescent-labeled probe 602 decreases. When the specimen solution is further heated, the stem-loop of the fluorescent-labeled probe 602 is also separated, so that the fluorescence intensity increases again. FIG. 6 shows an example of a result when a change in fluorescence intensity with respect to a change in change in temperature here is plotted on a graph. This fluorescent-labeled probe may be used together with a fluorescent-labeled probe for PCR, or a probe different from the fluorescent-labeled probe for PCR may be prepared and used. In addition, measurement of a change in fluorescence intensity with respect to a change in temperature may be performed in PCR, or may be performed by raising the temperature of the specimen solution independently of PCR (e.g. after completion of PCR).

In FIG. 6, the measurement result for the specimen solution a404 is shown in FIG. 6A, the measurement result for the specimen solution b405 is shown in FIG. 6C, the measurement result for the specimen solution c406 is shown in FIG. 6B, and the measurement result for the specimen solution d407 is shown in FIG. 6D. Further, when the fluorescence intensity changes in FIGS. 6A to 6D are differentiated by change in temperatures, the results shown in FIGS. 6E to 5H are obtained, respectively, and temperatures as inflection points are determined, and defined as the melting temperatures of a fluorescent-labeled probe and DNA for detecting a gene to be detected. In FIG. 4A, whether or not the specimen solution contains a gene to be detected cannot be determined from the measurement result for the specimen solution c406 and the specimen solution d407, but since the slope of the melting curve is large in FIG. 6B and small in FIG. 6D, the temperature difference between two points with a slope of a predetermined value on the melting curve is small in FIG. 6F and large in FIG. 6H, and when the temperature difference between two points with a slope of a predetermined value on the melting curve is plotted on the abscissa and the melting temperature is plotted on the ordinate as in FIG. 4B, it can be determined that the specimen solution c406 is a solution containing a wild-type of a target gene and the specimen solution d407 can is a solution containing both a wild-type and a mutant.

The melting temperature of the fluorescent-labeled probe for detecting a gene to be detected can be controlled by changing the sequence and strand length of the probe. In addition, the melting temperature can be controlled by using artificial DNA such as Peptide Nucleic Acid (PNA) or Locked Nucleic Acid (LNA). Depending on the design of a fluorescent-labeled probe, the melting temperatures of the wild-type and the mutant of a gene to be detected are significantly different, so that a gentle melting curve as in FIG. 6D is not obtained, the fluorescence intensity decreases in two stages, and both the peak in FIG. 6E and the peak in FIG. 6G may be observed when a differential curve is determined. Here, since the temperatures of both the wild-type and the mutant are obtained, the type of DNA in the specimen solution of each minute compartment can be determined by the melting temperature.

The combination of the fluorescent dye 603 and the quencher 604 of the fluorescent-labeled probe 602 used here is not particularly limited as long as it is a combination which is generally used for real-time PCR. Examples of the fluorescent dye 603 include FAM, VIC, ROX, Cy3 and Cy5, and examples of the quencher 604 include TAMRA, BHQ1, BHQ2 and BHQ3.

The sequence recognized by the fluorescent-labeled probe 602 may be on a gene identical to or different from a gene to be detected, and may be a gene having a sequence different by one base from the gene to be detected, e.g. a wild-type and a mutant of the same gene. As an example, when a genetic test for lung cancer is conducted, whether an ALK fusion gene and an EGFR gene mutation are present or not is determined for predicting the effect of a molecularly target drug. Here, the sequence may be a sequence that recognizes each of the ALK fusion gene and the EGFR gene, or may be a sequence that recognizes a L858R mutant of EGFR and a wild-type thereof.

(4) Method for Measuring Melting Temperature

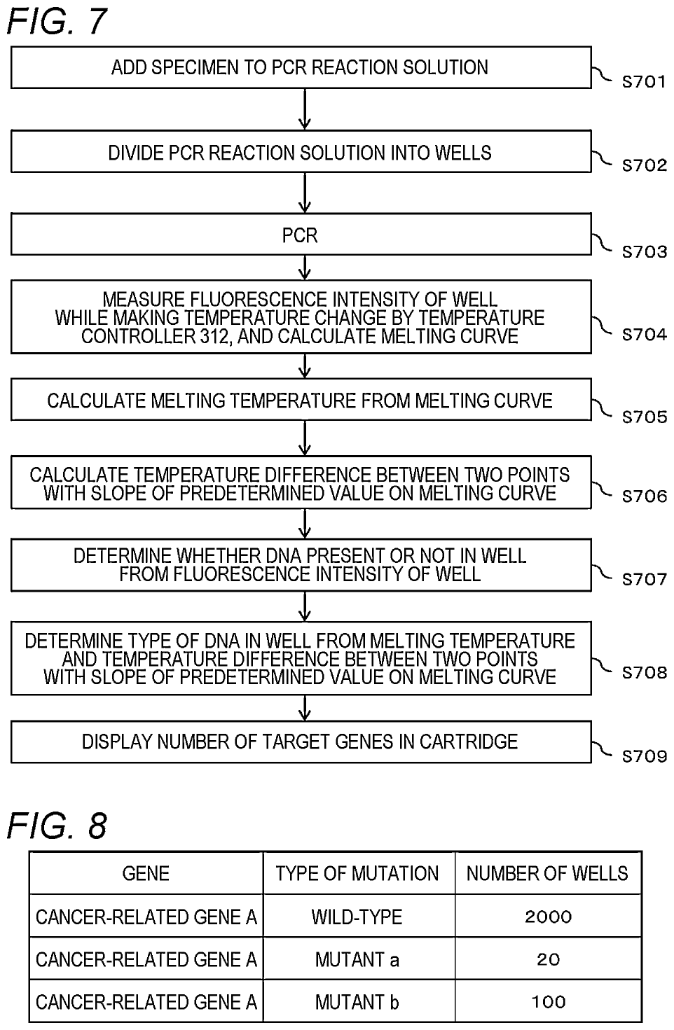

An example of a method for measuring the melting temperature using a cartridge including the device in FIG. 3B and the wells in FIG. 3D, and a DNA intercalator or a molecular beacon will be described with reference to the flowchart of FIG. 7. First, a specimen solution derived from a biological sample containing DNA is added to a PCR reaction solution containing a DNA polymerase, a primer, a DNA intercalator or a molecular beacon, a deoxyribonucleotide and a buffer solution (S701). The PCR reaction solution is divided into wells arranged on an array in the cartridge 313 (S702). The cartridge 313 is set in a thermal cycler, and PCR is carried out by temperature control of the thermal cycler (S703). The cycle of a denaturation step, an extension step and an annealing step is repeated to amplify DNA. The fluorescence intensity is increased by intercalation with the amplified DNA in the case of a DNA intercalator, and by hybridization with the amplified DNA in the case of molecular beacon. The reaction conditions such as the temperature, the time and the number of cycles in each step can be easily set by those skilled in the art. When the temperature is lowered to room temperature after PCR, the synthesized DNA forms a duplex.

After PCR, the cartridge 313 is placed on the temperature controller 312 of the DNA detector, the fluorescence measuring unit (FIG. 3A) measures the fluorescence intensity from the DNA intercalator or the molecular beacon in each well while the temperature of the cartridge 313 is changed by the temperature controller 312, and the obtained fluorescence data is sent to the calculation unit (not shown).

The calculation unit prepares a melting curve on the basis of the fluorescence data (S704), and calculates a melting temperature using the melting curve (S705). Further, a differential curve of the melting curve is prepared, and a temperature difference between two points with a slope of a predetermined value on the melting curve is calculated (S706). Whether DNA is present or not in the well is determined, where a well in which the fluorescence intensity is equal to or greater than the threshold is determined as being positive (having DNA), and a well in which the fluorescence intensity is equal to or less than the threshold is determined as being negative (having no DNA) (S707). For a well determined as being positive, the type of DNA in the well is determined from the melting temperature and the temperature difference between two points with a slope of a predetermined value on the melting curve (S708). Finally, the number of target genes in the cartridge is measured and displayed on a monitor.

When a change in fluorescence intensity in the well, which is associated with a change in temperature, is observed, a slope adjusting unit (not shown) may be provided under the temperature controller 312 on which the cartridge 313 is placed. The slope adjusting unit removes air bubbles generated in the cartridge 313 by a temperature from the temperature controller 312. This prevents a situation in which bubbles make acquirement of a fluorescence image impossible when the fluorescence intensity in each well is measured while the temperature of the sample is lowered by the temperature controller 312.

In the determination of whether the DNA in each well is positive or negative, information on the fluorescence intensity is used. Here, the fluorescence intensity can be standardized by using, for example, a ratio or a difference between the fluorescence intensity at a temperature lower than the melting temperature and the fluorescence intensity at a temperature higher than the melting temperature. By, for example, subtracting the fluorescence intensity at 85° C. from the fluorescence intensity at 50° C., an impact of fluorescence of the fluorescent-labeled probe itself, i.e., an impact of background can be removed.

A predetermined threshold of the fluorescence intensity, a predetermined range of the melting temperature and a threshold of the temperature difference between the two points with a slope of a predetermined value on the melting curve may be statistically determined by an operator from the results of a pilot experiment or the like conducted in advance, or may be determined automatically. In addition, for each digital PCR measurement, the threshold of the fluorescence intensity and the predetermined range of the melting temperature may be statistically determined using the measurement data in each well in the cartridge.

The data for statistically discriminating DNA in the well may include the following items: a fluorescence intensity at a temperature lower than the melting temperature; a fluorescence intensity at a temperature higher than the melting temperature; a ratio of a fluorescence intensity at a temperature lower than the melting temperature to a fluorescence intensity at a temperature higher than the melting temperature; a difference between a fluorescence intensity at a temperature lower than the melting temperature and a fluorescence intensity at a temperature higher than the melting temperature; a melting temperature; a characteristic amount representing a shape of a melting curve; and the like.

The specimen solution to be used is not particularly limited, and may be a sample containing DNA to be detected. Examples thereof include biological samples such as body fluids, tissues, cells and excretions of animals and plants, and samples containing fungi, bacteria and the like, such as soil samples. Examples of the body fluid include blood, saliva, and cerebrospinal fluid, and the blood contains cell free DNA (cf DNA) and circular tumor DNA (ct DNA) present therein. Examples of the tissue include disease-affected parts (e.g., cancer tissues in the breast, the liver and the like) obtained by surgical operations or biopsy techniques. The tissue may be an already fixed tissue, e.g. a formalin-fixed paraffin-embedded tissue section (FFPE). Examples of the cell include cells collected at or near affected parts, and circular tumor cells circulating in blood. The pretreatment of these specimens is not particularly limited, and those obtained by collecting a sample from a living body, the environment or the like, then adding the sample to suspension and homogenizing the mixture or dissolving the sample with a lytic liquid may be used directly, and it is preferable to use those obtained by extracting or purifying the nucleic acid contained in the sample.

It is desirable that oil be added to the upper surface of a PCR reaction solution so that the PCR reaction solution divided into wells does not evaporate during measurement of PCR and melting curve analysis. The oil is preferably a substance which is insoluble or hardly soluble in the PCR reaction solution and chemically inactive and which is stable to a change in temperature at a high temperature as in PCR. Fluorine-based oil, silicone-based oil, hydrocarbon-based oil and the like can be used. Examples of the fluorine-based oil include perfluorocarbon and hydrofluoroether. Fluorine-based oil having longer carbon chain is more preferable because it has lower volatility. Examples of the silicone-based oil include polyphenylmethylsiloxane and trimethylsiloxysilicate. Examples of the hydrocarbon-based oil include mineral oil, liquid paraffin and hexadecane. A surfactant may be added to the oil before use. Here, the type of surfactant is not particularly limited, and Tween 20, Tween 80, Span 80, Triton X −100 and the like can be applied.

(5) Display of Result

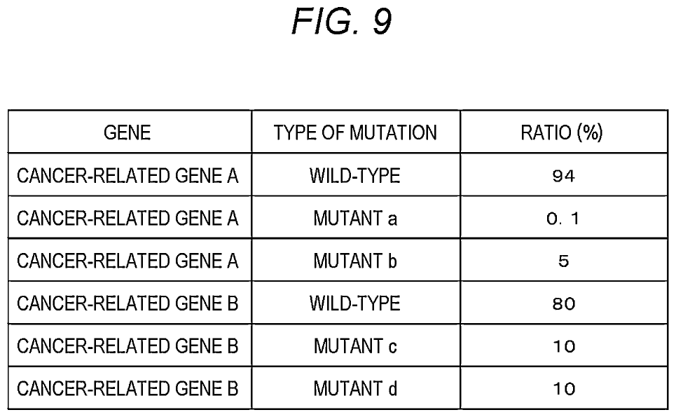

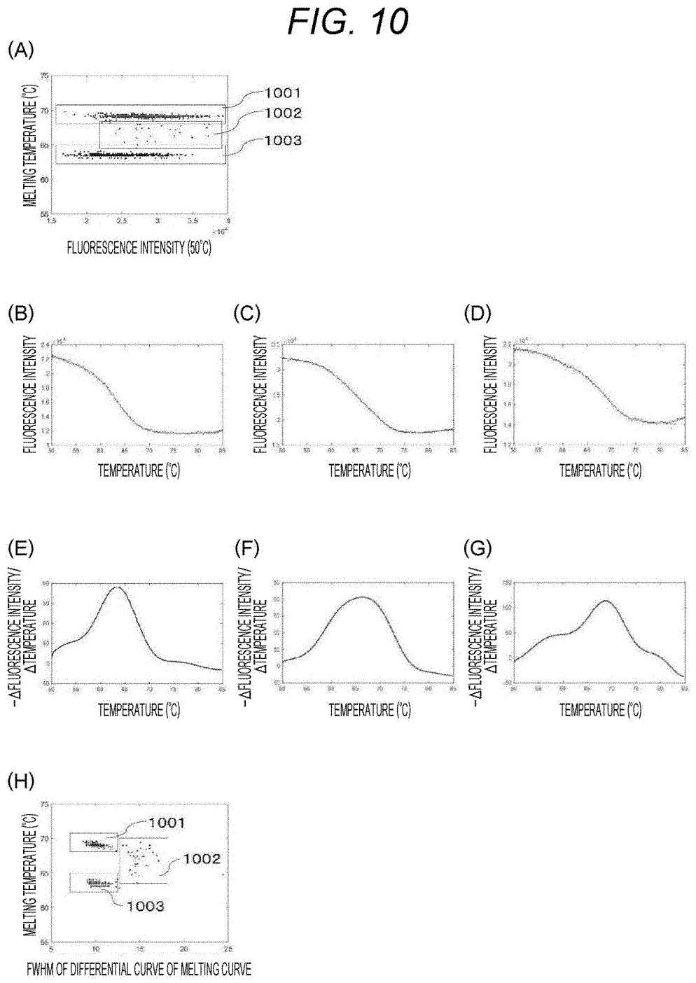

FIGS. 8 and 9 show an example of images of measurement results displayed on the monitor. As shown in FIG. 8, the number of specimen solutions counted for each type of oncogene or each type of mutation may be displayed, or as shown in FIG. 9, the ratio of specimen solutions counted for each type of oncogene or each type of mutation may be displayed. The results displayed on the monitor may include not only the number and the ratio of the specimen solutions as shown in FIG. 8 and FIG. 9 but also a graph obtained by plotting measured values of the specimen solution on two axes representing a temperature difference between two points with a slope of a predetermined value on the melting curve and a melting temperature as shown in FIG. 1. The results may also include a histogram obtained by plotting the number of specimen solutions with respect to the fluorescence intensity or melting temperature of the fluorescent-labeled probe. The user can also observe the graph or the histogram, and change the ranges of the fluorescence intensity and the melting temperature of the fluorescent-labeled probe and/or the range of the temperature difference between two points with a slope of a predetermined value on the melting curve, followed by counting again the number of specimen solutions in which the fluorescence intensity and the melting temperature fall within the ranges.

As described above, the specimen solution is treated as a solution in wells or droplets, and therefore the number of specimen solutions may be replaced by the number of wells or the number of droplets.

(6) Program

One embodiment of the present invention is a program for causing a DNA detector to carry out a method for detecting DNA. Here, the device described in detail in (2) is used as the DNA detector, and the method described in detail in (1) is carried out as the method for detecting DNA.

A recording medium which stores the program is also one of embodiments of the present invention.

### EXAMPLE

This example shows the results of measuring the melting temperature of DNA in a well using a fluorescent-labeled probe.

First, genomic DNAs of a wild-type and a G13D mutant of a KRAS gene (final concentration: 133 molecules/μL) were prepared, and a forward primer (final concentration: 0.25 μM), a reverse primer (final concentration: 2.0 μM), a fluorescent-labeled probe corresponding to the wild-type (final concentration: 0.5 μM), a fluorescent-labeled probe corresponding to the G13D mutant (final concentration: 0.5 μM) and a 1× master mix (DNA polymerase, including dNTPs), which are required for PCR, were added to a PCR reaction solution. Here, the primer pair was added in such a manner that the concentrations of the primer pair were asymmetric so as to excessively amplify the complementary DNA strand of the fluorescent-labeled probe. The sequences of the primer and the probe are as follows. All of the fluorescent-labeled probes are designed such that complementary sequences are present near both ends, and form a double strand in the molecule. In addition, HEX as a fluorescent dye is bound to the 5′ end, and BHQ-1 as a quencher is bound to the 3′ end.

Forward primer:
(SEQ ID NO: 1)
5′-GTCACATTTTCATTATTTTTATTATAAGG-3′
Reverse primer:
(SEQ ID NO: 2)
5′-GTATCGTCAAGGCACTCTTGCC-3′
Fluorescent-labeled probe corresponding to wild-
type:
(SEQ ID NO: 3)
5′-TTGGAGCTGGTGGCGT-3′
Fluorescent-labeled probe corresponding to mutant:
(SEQ ID NO: 4)
5′-CTGGTGACGTAGGCA-3′

Thereafter, 15 μL of the PCR reaction solution was added to each well in such a manner that one of the wild-type DNA and the G13D mutant DNA of KRAS gene was present or either of the DNAs was not present in the well, and DNA was amplified by PCR. The PCR reaction was carried out by performing treatment at 96° C. for 10 minutes, then performing cycles of (60° C., 2 minutes→98° C., 30 seconds), and finally performing treatment at 60° C. for 2 minutes. After the reaction, measurement and analysis of the melting curve were performed by observing a change in fluorescence intensity in each well while cooling the chip provided with the well from 85° C. to 50° C. on a temperature control stage.

FIG. 10A shows the results of measuring specimens obtained by mixing equal amounts of a wild-type and a G13D mutant of KRAS gene, where the fluorescence intensity at 50° C. is plotted on the abscissa and the melting temperature is plotted on the ordinate. A difference in melting temperature caused division into two distributions. A population 1001 having a distribution around 69° C. corresponds to wells containing only the wild-type, and a population 1003 having a distribution around 63° C. corresponds to wells containing only the mutant. Points 1002 where the melting temperature widely ranges from 63° C. to 69° C. are wells containing 1 copy of each of the wild-type and the G13D mutant.

The melting curves for wells included in the populations 1001, 1002 and 1003 divided by the melting temperature in FIG. 10A are shown in FIGS. 10B to 10D, respectively, and the differential curves of the melting curve are shown in FIGS. 10E to 10G. In the wells containing only the wild-type and the wells containing only the G13D mutant, the slope of the melting curve is large as shown in FIGS. 10B and 10D, and the FWHM (full width at half maximum) of the differential curve is small as shown in FIGS. 10E and 10G. On the other hand, it is apparent that in the well containing one copy of each of the wild-type and the G13D mutant, the slope of the melting curve is small and the FWHM (full width at half maximum) of the differential curve is large as shown in FIG. 10C.

FIG. 10H shows the results of measuring specimens obtained by mixing equal amounts of a wild-type and a G13D mutant of KRAS gene, where the FWHM (full width at half maximum) of the differential curve of the melting curve is plotted on the abscissa and the melting temperature is plotted on the ordinate. As a result of using the FWHM (full width at half maximum) of the differential curve of the melting curve as the abscissa, the well containing one type of KRAS gene and the well containing two types of KRAS genes are different in distribution in the abscissa direction, and therefore it is possible to clearly discriminate the populations of wells containing only the wild-type, wells containing only the G13D mutant, and wells containing two types which are the wild-type and the G13D mutant.

Thus, by using the slope of the melting curve and the FWHM (full width at half maximum) of the differential curve of the melting curve for discrimination of the type of DNA in the well, a well which contains one copy of each of the wild-type and the mutant and cannot be discriminated by only the fluorescence intensity and the melting temperature, can be discriminated, so that it is possible to improve measurement reproducibility and measurement accuracy.

### INDUSTRIAL APPLICABILITY

According to the present invention, it is possible to provide a novel digital PCR analysis method for clearly discriminating minute compartments with two different types of target genes present in one compartment by a measurement device and correcting the count number of target genes in digital PCR using melting curve analysis.

### REFERENCE SIGNS LIST

101 minute compartment containing wild-type gene
102 minute compartment containing mutant gene
103 minute compartment containing wild-type and mutant gene
201 minute compartment containing wild-type gene
202 minute compartment containing mutant gene
203 minute compartment containing wild-type and mutant gene
301 droplet containing target gene
302 droplet free of target gene
303 microchannel
304 light source
305 filter
306 photomultiplier
307 CCD
308 lens
309 dichroic mirror
310 droplet detection cartridge
311 droplet
312 temperature controller
313 well-type detection cartridge
314 well containing target gene
315 well free of target gene
401 minute compartment containing wild-type gene
402 minute compartment containing mutant gene
403 minute compartment containing wild-type and mutant gene
404 minute compartment a
405 minute compartment b
406 minute compartment c
407 minute compartment d
501 DNA
502 DNA intercalator
503 melting temperature
601 DNA
602 fluorescent-labeled probe
603 fluorescent dye
604 quencher
605 melting temperature
1001 well containing only wild-type
1002 well containing one copy of each of wild-type and
mutant
1003 well containing only mutant

1. A method for detecting DNA by digital PCR, comprising the steps of:
dividing a DNA solution containing a plurality of types of DNAs to be detected and a plurality of types of fluorescent-labeled probe into a plurality of compartments, the plurality of types of fluorescent-labeled probe being labeled with a same fluorescent and each of the plurality of types of fluorescent-labeled probe binding to respective of the plurality of types of DNAs;
carrying out PCR in the compartments; measuring a fluorescence intensity in association with a change in temperature; calculating a melting temperature of a DNA double strand from a change in fluorescence intensity, which is associated with a change in temperature of the DNA solution; and
calculating a temperature difference between two points with a slope of a predetermined value on a melting curve indicating a change in the fluorescence intensity, wherein the difference of the melting temperature of each of the plurality of types of fluorescent-labeled probe and the melting temperature of the respective of the plurality of types of DNAs is 10° C. or less.

2. The method for detecting DNA according to claim 1, further comprising the step of identifying a compartment, in which the temperature difference is equal to or greater than a predetermined threshold, as a compartment containing two types of DNAs to be detected.

3. The method for detecting DNA according to claim 1, further comprising the step of identifying a compartment, in which the temperature difference is less than a predetermined threshold, as a compartment containing one type of DNA to be detected.

4. The method for detecting DNA according to claim 1, wherein the DNA solution contains the fluorescent-labeled probe, and the melting temperature is a melting temperature of a double strand formed between the fluorescent-labeled probe and the DNA to be detected.

5. The method for detecting DNA according to claim 4, wherein the fluorescent-labeled probe has a fluorescent dye and a quencher thereof.

6. (canceled)

7. The method for detecting DNA according to claim 1, wherein the plurality of compartments are arranged in a plane.

8. The method for detecting DNA according to claim 1, wherein the DNA solution is divided into the plurality of compartments by droplets or wells.

9. A DNA detector for detecting DNAs by digital PCR in a DNA solution, the DNA detector comprising:
a heating unit for heating the DNA solution, the DNA solution containing a plurality of types of DNAs to be detected and a plurality of types of fluorescent-labeled probe, the plurality of types of fluorescent-labeled probe being labeled with the same fluorescent and each of the plurality of types of fluorescent-labeled probe binding to respective of the plurality of types of DNAs, wherein the difference of the melting temperature of each of the plurality of types of fluorescent-labeled probe and the melting temperature of the respective of the plurality of types of DNAs is 10° C. or less;
an amplification unit for amplifying the plurality of types of DNAs to be detected;
a fluorescence measuring unit for measuring an intensity of fluorescence emitted from the DNA solution; and
a calculation unit for calculating a melting temperature of a DNA double strand from a change in intensity of the fluorescence, which is associated with a change in temperature of the DNA solution, and calculating a temperature difference between two points with a slope of a predetermined value on a melting curve indicating the change in the fluorescence intensity.

10. (canceled)

11. The DNA detector according to claim 9, further comprising a monitor which displays the detection result.

12. A computer program product for causing a DNA detector to carry out the method for detecting DNA according to claim 1.

13. The computer program product according to claim 12, wherein the DNA detector is a DNA detector for detecting DNAs by digital PCR in a DNA solution, the DNA detector comprising:
a heating unit for heating the DNA solution, the DNA solution containing a plurality of types of DNAs to be detected and a plurality of types of fluorescent-labeled probe, the plurality of types of fluorescent-labeled probe being labeled with the same fluorescent and each of the plurality of types of fluorescent-labeled probe binding to respective of the plurality of types of DNAs, wherein the difference of the melting temperature of each of the plurality of types of fluorescent-labeled probe and the melting temperature of the respective of the plurality of types of DNAs is 10° C. or less;
an amplification unit for amplifying the plurality of types of DNAs to be detected;
a fluorescence measuring unit for measuring an intensity of fluorescence emitted from the DNA solution; and
a calculation unit for calculating a melting temperature of a DNA double strand from a change in intensity of the fluorescence, which is associated with a change in temperature of the DNA solution, and calculating a temperature difference between two points with a slope of a predetermined value on a melting curve indicating the change in the fluorescence intensity.

14. A non-transitory recording medium which stores the computer program product according to claim 12.
